(19) United States
(12) Patent Application Publication
(10) Pub. No.: US 2014/0288530 A1
(43) Pub. Date: Sep. 25, 2014

(54) SYSTEM AND METHOD FOR ADAPTABLE SAFETY SYRINGE

(71) Applicant: Reprise Technologies, LLC., Menlo Park, CA (US)

(72) Inventors: Alan E. Shluzas, San Carlos, CA (US); Stephen H. Diaz, Palo Alto, CA (US); John F. Shanley, Emerald Hills, CA (US)

(73) Assignee: Reprise Technologies, LLC., Menlo Park, CA (US)

(21) Appl. No.: 14/222,486

(22) Filed: Mar. 21, 2014

Related U.S. Application Data
(60) Provisional application No. 61/804,604, filed on Mar. 22, 2013.

(51) Int. Cl. A61M 5/32; A61M 39/10

(52) U.S. Cl. CPC A61M 5/322; A61M 39/10; A61M 2039/1077
USPC 604/506; 604/218

(57) ABSTRACT

One embodiment is directed to a method of conducting an injection, comprising: providing an assembly of a syringe plunger movably coupled to a syringe body which may contain fluid for infusion, the syringe body having a distal end that is removably coupled to a Luer adaptor, the Luer adaptor defining a needle lumen through which a needle may be coupled, the Luer adaptor comprising a distal surface configured to be removably coupled to a Luer fitting of another device; deciding upon infusion via needle injection or Luer coupling; and selectably configuring the assembly for needle injection or Luer coupling by removing the Luer adaptor to further expose the needle for needle injection, or by leaving the Luer adaptor in place and coupling it to the Luer fitting of the other device.

### RELATED APPLICATION DATA

The present application claims the benefit under 35 U.S.C. §119 to U.S. provisional patent application Ser. No. 61/804,604, filed Mar. 22, 2013. The foregoing application is hereby incorporated by reference into the present application in its entirety.

### FIELD OF THE INVENTION

The present invention relates generally to injection systems, devices, and processes for facilitating various levels of control over fluid infusion, and more particularly to systems and methods related to safety syringes in an environment wherein both Luer adaptation and hypodermic needle adaptation may be desired.

### BACKGROUND

Millions of syringes, such as that depicted in FIG. 1A (2), are consumed in healthcare environments every day. A typical syringe (2) comprises a tubular body (4), a plunger (6), and an injection needle (8). As shown in FIG. 1B, such a syringe (2) may be utilized not only to inject fluid into a patient, but also to withdraw or expel fluid out of or into a container such as a medicine bottle or vial (10). Indeed, due to regulatory constraints in some countries such as the United States, upon use of a medicine bottle (10) with a syringe (2) as shown in a particular patient's environment, such medicine bottle may only be utilized with that patient and then must be disposed of—causing significant medical waste from bottle and remaining medicine disposal, and even contributing to periodic shortages of certain critical drugs. Referring to FIG. 2A, three Luer-type syringes (12) are depicted, each having a Luer fitting geometry (14) disposed distally, so that they may be coupled with other devices having similar mating geometry, such as the Luer manifold assembly (16) depicted in FIG. 2B. The Luer fittings (14) of the syringes of FIG. 2A may be termed the “male” Luer fittings, while those of FIG. 2B (18) may be termed the “female” Luer fittings; they may be coupled by relative rotation, which may be combined with compressive loading, to engage threads within the male fitting which are configured to engage a flange on the female fitting and bring the devices together into a fluid-sealed coupling. While such Luer coupling is perceived to be relatively safe for operators, there is risk of medicine spilling and parts breakage during the loading to provide a Luer coupling. The use of needle injection configurations, on the other hand, carries with it the risk of a sharp needle contacting or poking a structure that is not desired. For this reason, socalled “safety syringes” have been developed. One embodiment of a safety syringe (20) is shown in FIG. 3, wherein a tubular shield member (22) is spring biased to cover the needle (8) when released from a locked position relative to the syringe body (4). Another embodiment of a safety syringe (24) is shown in FIGS. 4A-4B. With such a configuration, after full insertion of the plunger (6) relative to the syringe body (4), the retractable needle (26) is configured to retract (28) back to a safe position within the tubular body (4), as shown in FIG. 4B. One of the challenges with such a configuration is that there is a chance that the retracted needle (26) may become realigned with the exit port out of the tubular body (4), such that it may be re-inserted back out to an exposed needle condition. Referring to FIG. 5, a Luer style syringe (12) may be coupled (30) to a needle having a Luer fitting to convert the syringe for direct injection usage—but such a coupling generally requires manual manipulation of the needle (8) and/or female Luer fitting (19) coupled thereto, which may involve undesired healthcare provider risk.

There is a need for improved injection systems which address the shortcomings of currently-available configurations.

### BRIEF DESCRIPTION OF THE DRAWINGS

FIGS. 1A-B, 2A-B 3,4A-B and 5 illustrate various aspects of conventional injection syringe configurations.

FIGS. 6A-6C illustrate various aspects of one embodiment of an adaptable safety syringe assembly in accordance with the present invention, wherein the syringe body comprises a glass material.

FIGS. 7A-7C illustrate various aspects of one embodiment of an adaptable safety syringe assembly in accordance with the present invention, wherein the syringe body comprises a polymeric material.

FIG. 8 illustrates various aspects of one embodiment of a process for conducting an injection using an adaptable safety syringe configuration in accordance with the present invention.

FIG. 9A-9C illustrate various aspects of one embodiment of an adaptable safety syringe assembly in accordance with the present invention, including a removable outer coupling jacket.

FIG. 10 illustrates various aspects of one embodiment of a process for conducting an injection using an adaptable safety syringe configuration in accordance with the present invention.

FIGS. 11A-11D illustrate various aspects of one embodiment of an adaptable safety syringe assembly in accordance with the present invention, wherein the needle is reoriented upon retraction to prevent further insertion.

FIGS. 12A-12G illustrate various aspects of one embodiment of an adaptable safety syringe assembly in accordance with the present invention, wherein the needle is reoriented upon retraction to prevent further insertion.

FIGS. 13A-13B illustrate various aspects of one embodiment of an adaptable safety syringe assembly in accordance with the present invention, wherein the needle is reoriented upon retraction to prevent further insertion.

FIG. 14 illustrates various aspects of one embodiment of a process for conducting an injection using an adaptable safety syringe configuration in accordance with the present invention.

FIGS. 15A-15C illustrate aspects of one embodiment of a Luer adaptor having a finger protection flange.

FIG. 16 illustrates various aspects of one embodiment of a process for conducting an injection using an adaptable safety syringe configuration in accordance with the present invention.

### SUMMARY OF THE INVENTION

One embodiment is directed to a method of conducting an injection, comprising: providing an assembly of a syringe plunger movably coupled to a syringe body which may contain fluid for infusion, the syringe body having a distal end that is removably coupled to a Luer adaptor, the Luer adaptor defining a needle lumen through which a needle may be coupled, the Luer adaptor comprising a distal surface configured to be removably coupled to a Luer fitting of another device; deciding upon infusion via needle injection or Luer coupling; and selectably configuring the assembly for needle injection or Luer coupling by removing the Luer adaptor to further expose the needle for needle injection, or by leaving the Luer adaptor in place and coupling it to the Luer fitting of the other device. The method further may comprise inserting the plunger relative to the syringe body to inject fluid into an infusion target. With Luer coupling selected, the method further may comprise rotatably coupling the distal surface of the Luer adaptor to the Luer fitting of the other device. With needle injection selected, the method further may comprise inserting a portion of the needle directly into a patient. The method further may comprise coupling a proximal end of the needle to the plunger distal end upon full insert of the plunger relative to the syringe body. The method further may comprise reorienting the needle upon full retraction relative to the syringe body to avoid re-insertion of the needle relative to the syringe body. The method may comprise reaching full retraction by pulling the plunger relative to the syringe body. Reaching the state of full retraction may be assisted via a vacuum load developed within the syringe body upon insertion of the plunger relative to the body. Removing the Luer adaptor may comprise removing an outer coupling jacket configured to retain coupling between the Luer adaptor and the syringe body. Removing the outer coupling jacket may comprise manually manipulating a portion of the outer coupling jacket that is pre-biased to tear upon loading for easy removal.

Another embodiment is directed to a safety syringe system, comprising: a plunger movably coupled to a syringe body, the syringe body having proximal and distal ends; and a Luer adaptor removably coupled to the distal end of the syringe body and defining a needle lumen through which a needle may be coupled, the Luer adaptor comprising a distal surface configured to be removably coupled to a Luer fitting of another device; wherein the needle has proximal and distal ends, the distal end configured for direct injection into an injection target structure, the proximal end configured to be coupled to the plunger such that upon withdrawal after insertion, the needle is pulled into and contained by the syringe body. The syringe body may comprise a glass construct. In another embodiment the syringe body may comprise a polymeric construct. The distal surface may comprise a Luer mating geometry. The other device may be removably coupled to the distal surface by inducing relative rotational motion between the other device and the distal surface. The distal end of the needle may comprise a hypodermic needle tip. The proximal end of the needle may be configured to be compressibly coupled to the plunger. The system further may comprise an outer coupling jacket configured to retain the coupling between the Luer adaptor and the syringe body when in place. The outer coupling jacket may be removably coupled across portions of both the Luer adaptor and the syringe body. The outer coupling jacket may comprise a heat shrink tubing member.

### DETAILED DESCRIPTION

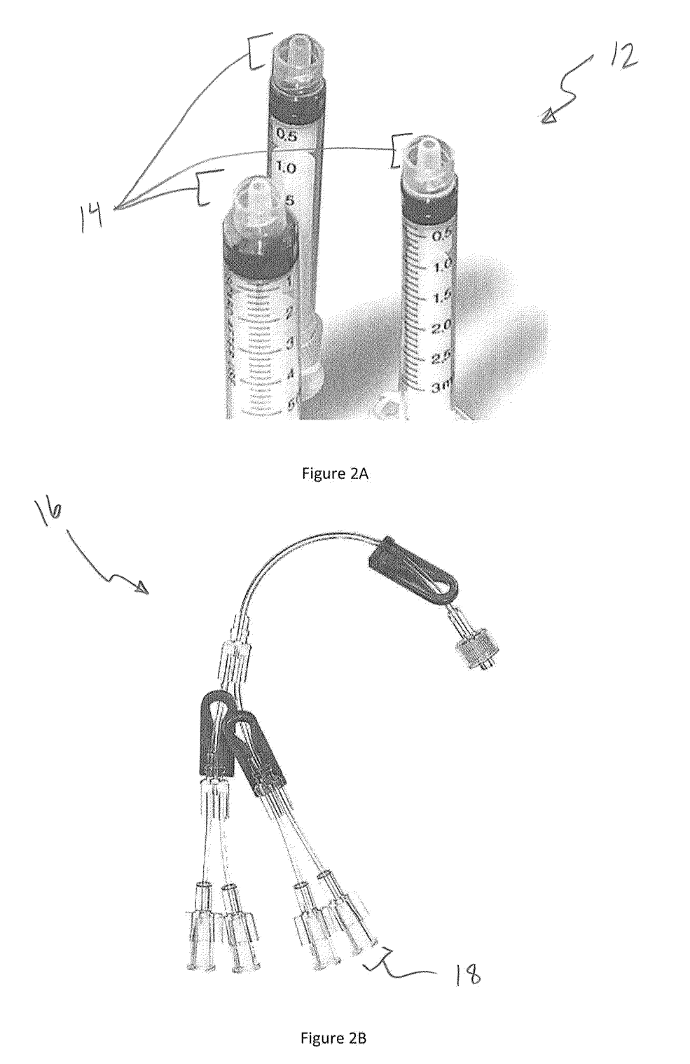

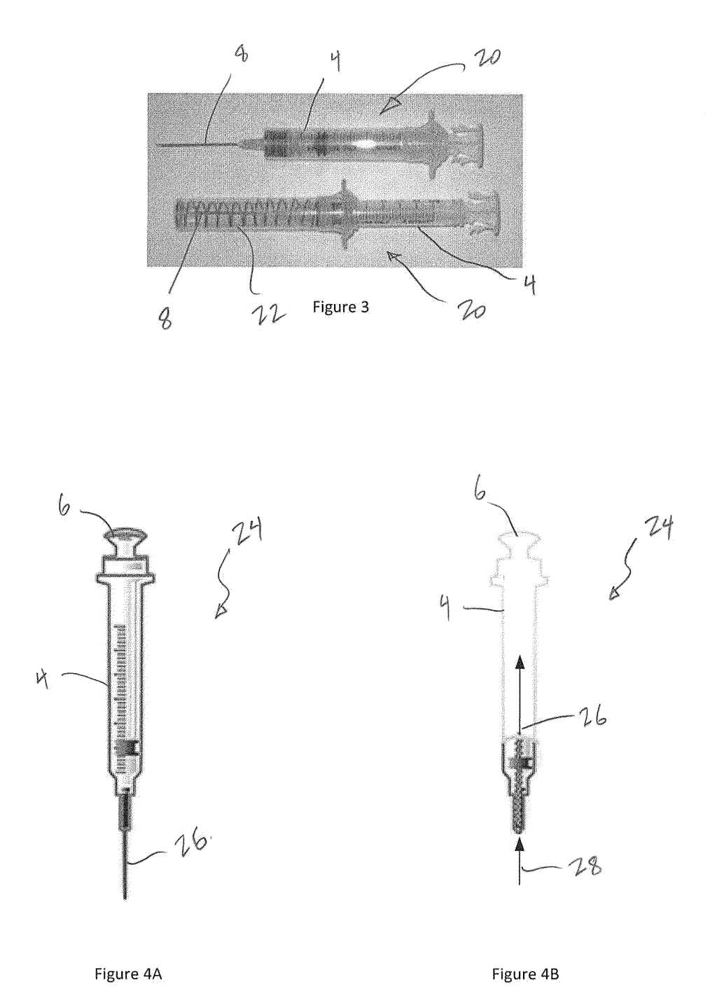

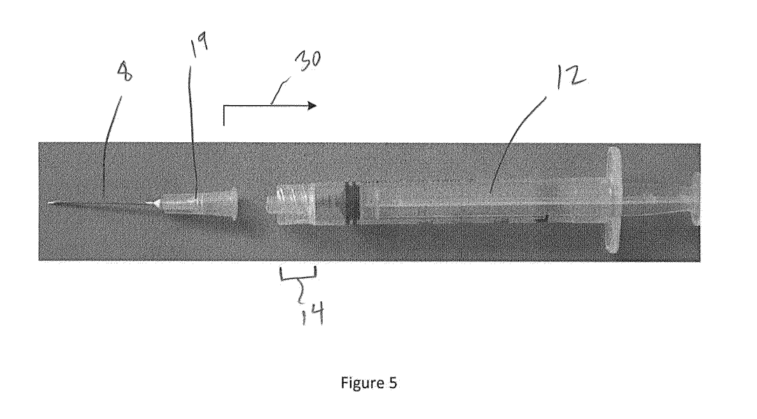

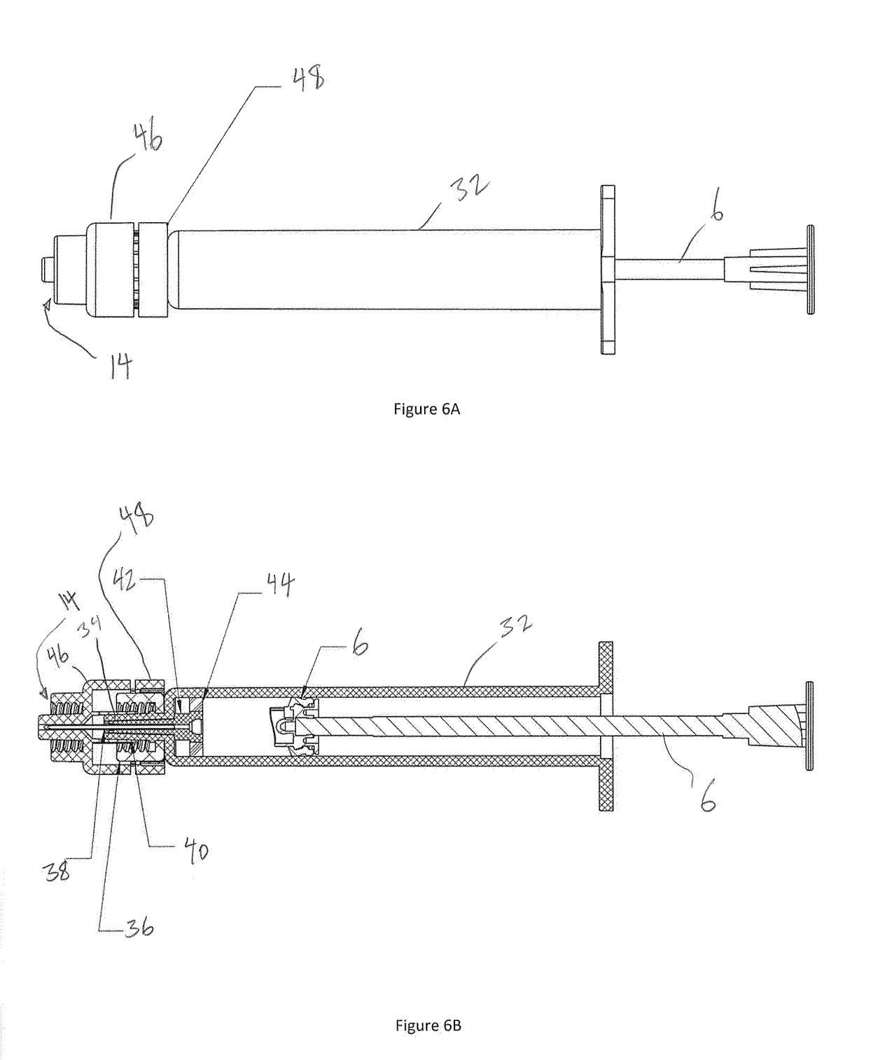

Referring to FIGS. 6A-6C, a safety syringe embodiment is depicted wherein a Luer adaptor allows for convenient switching between a direct needle injection configuration—and a Luer adaptor injection configuration wherein the needle remains safety contained. Referring to FIG. 6A, a glass syringe body (32) is shown with a plunger (6) inserted therein. The distal portion of the body (32) is coupled to a Luer adaptor coupling member (48), which is removably coupled to a Luer adaptor member (46), the distal portion of which comprises a Luer interface (14) for Luer style injection coupling as described above. FIG. 6B shows a cross sectional view wherein in the Luer configuration with the Luer adaptor member intact, as illustrated in FIG. 6A, the needle member (38) is safely contained within a lumen or passageway formed through the distal portion (34) of the syringe body (32) for a Luer-configuration injection fitting without using the needle (38). A Luer fitting (36) is bonded, such as with an adhesive, to the distal portion (34) of the syringe body (32) to provide a threaded interface for coupling the Luer adaptor (46) to the syringe body (32). In the event that conversion to needle injection is desired, an operator may manually apply a twisting load to the Luer adaptor (46), thereby utilizing the threads of the Luer adaptor (46) to controllably break or fracture a frangible interface between the Luer adaptor (46) and the Luer adaptor coupling member (48), which is fixedly attached to the Luer fitting (36) and therefore to the syringe body (32). Thus such a twisting motion allows for controlled removal of the Luer adaptor (46), and therefore exposure of the needle (38) for direct needle injection. When remaining in the Luer coupling configuration, such as is shown in FIGS. 6A and 6B, a tapered fluid seal (40) seals the interface between Luer adaptor (46) and syringe body (32) distal portion (34).

Referring to FIG. 6C, a partially exploded orthogonal view of a configuration such as is shown in FIGS. 6A and 6B is depicted, also illustrating that this safety syringe configuration includes a needle (38) which is coupled to the distal portion of the syringe body (32) (and held in place and sealed by a grommet member 44) until the plunger (6) is fully inserted into the syringe body, wherein a needle-mating fitting on the plunger couples to the needle holder structure (42) and is configured to withdraw the needle (38) back into the body (32) of the syringe upon withdrawal of the plunger (6) relative to the syringe body (32).

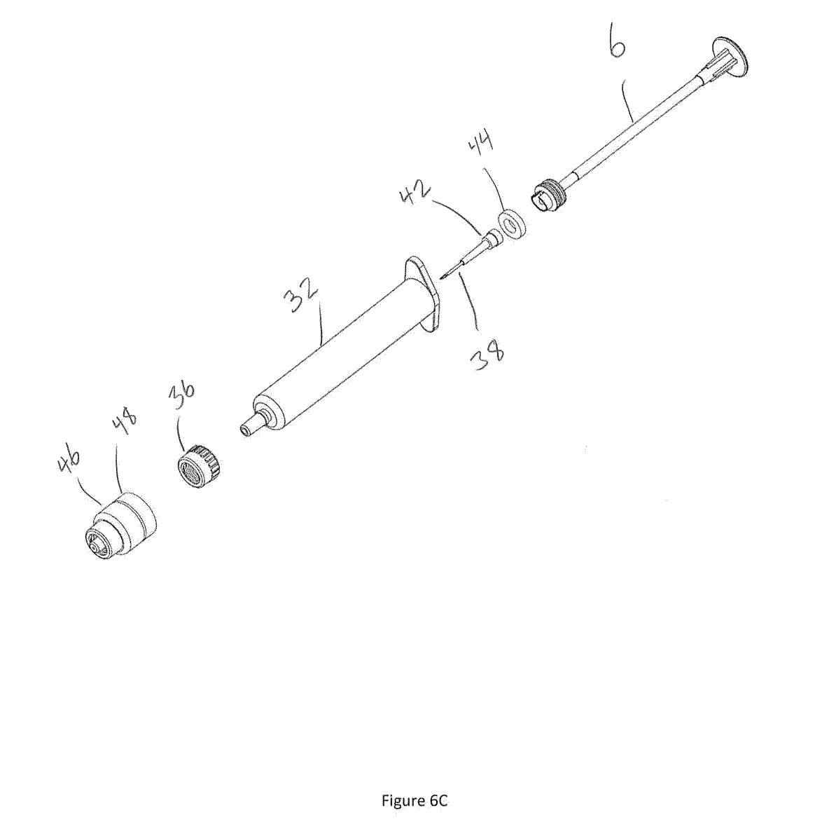

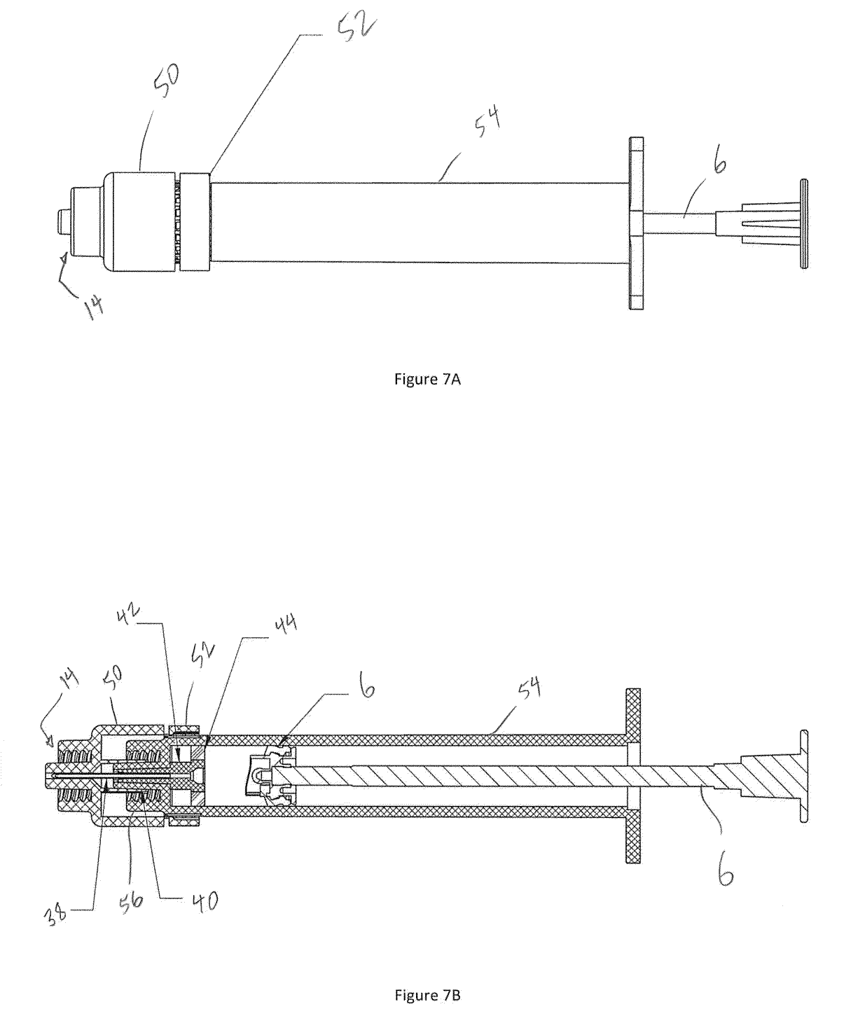

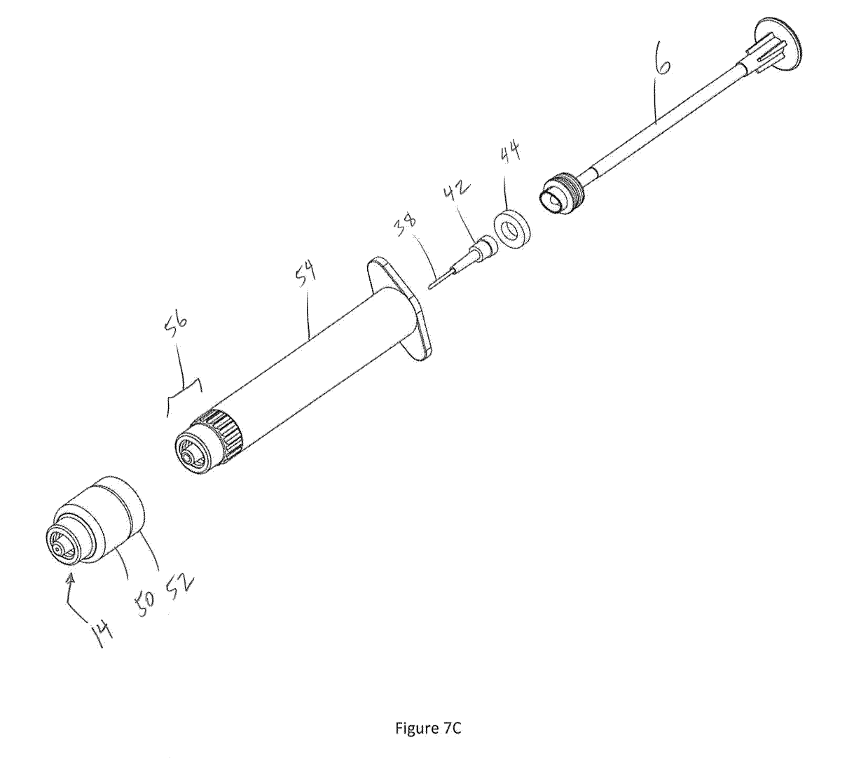

FIGS. 7A-7C illustrate a similar configuration to that depicted in FIGS. 6A-6C, with the exception that the syringe body (54) comprises a polymeric material into which geometric features may be more easily formed as compared with glass. As shown in FIG. 7C, the embodiment of FIGS. 7A-7C features a Luer fitting (56) that may be formed into the distal portion of the syringe body (54) so that a separate Luer fitting (such as element 36 in FIGS. 6A-6C, which may be bonded to a glass syringe body distal portion as described above) is not required. As shown in FIG. 7B, the Luer adaptor coupling member (52) may be mounted directly to the Luer fitting (56), and may be removably coupled to the Luer adaptor (50) via a frangible coupling interface that may be controllably broken or released with relative motion, as described above.

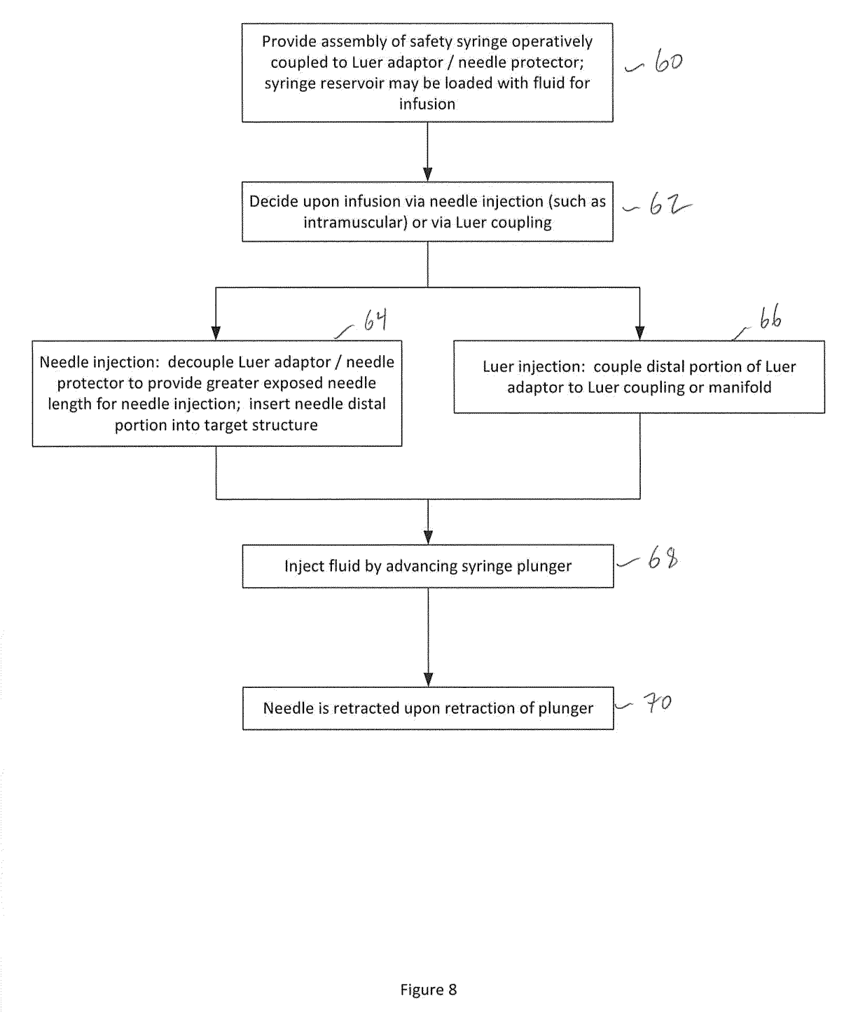

Referring to FIG. 8, configurations such as those described in reference to FIGS. 6A-7C may be utilized in operation to provide an assembly having a controllably removable Luer adaptor (60). The assembly may be prepackaged in kits with a precision amount of medication or other fluid measured into the volume between the plunger and syringe body such that a full insertion of the plunger always receives a predicted and premeasured amount of the medication or other fluid. For example, in one embodiment, pre-measured assemblies are available in varieties of medications at 5 milliliter intervals. The healthcare provider may decide upon infusion via either needle or Luer coupling (62), and then the assembly may be transformed appropriately: with needle injection (64) the Luer adaptor may be removed at the frangible coupling with the coupling member to expose the injection needle and allow for direct injection of the injection target (i.e., a muscle, etc); with Luer injection (66), the distal Luer interface may be utilized for simple Luer coupling while the needle remains in place, protected and unexposed from the operator within the housing provided by the intact Luer adaptor and other portions of the assembly, as shown, for example, in FIGS. 6A-7C. Injection may be completed by advancing the plunger relative to the syringe body (68), and with the safety syringe style needle coupling to the plunger upon full insertion of the plunger, the needle may be retracted back into a protected configuration, wherein it is at least partially housed within the syringe body (70).

Referring to FIGS. 9A-9C, a configuration similar to that of FIGS. 6A-6C is depicted, with the exception that a removable adaptor coupling jacket (78) has been coupled and fitted around the decouplable junction between the Luer adaptor (46) and the Luer adaptor coupling member (element 48 in FIG. 6A, for example) to bolster this removable coupling and ensure that it does not become accidentally uncoupled. To controllably decouple the Luer adaptor (46) from the Luer adaptor coupling member, first the removable adaptor coupling jacket (78) must be removed, after which the Luer adaptor (46) may be twisted off, breaking the frangible coupling interface between it and the Luer adaptor coupling member. In the depicted embodiment, the removable adaptor coupling jacket (78) comprises a manipulation tab (82) configured to allow an operator to pull along a pre-biased tearing or perforation pattern (80) to controllably release the jacket (78), which may comprise a polymeric material such as polyethylene or heat shrink tubing material. FIG. 9C illustrates an exploded view of a configuration such as that featured in FIGS. 9A and 9B.

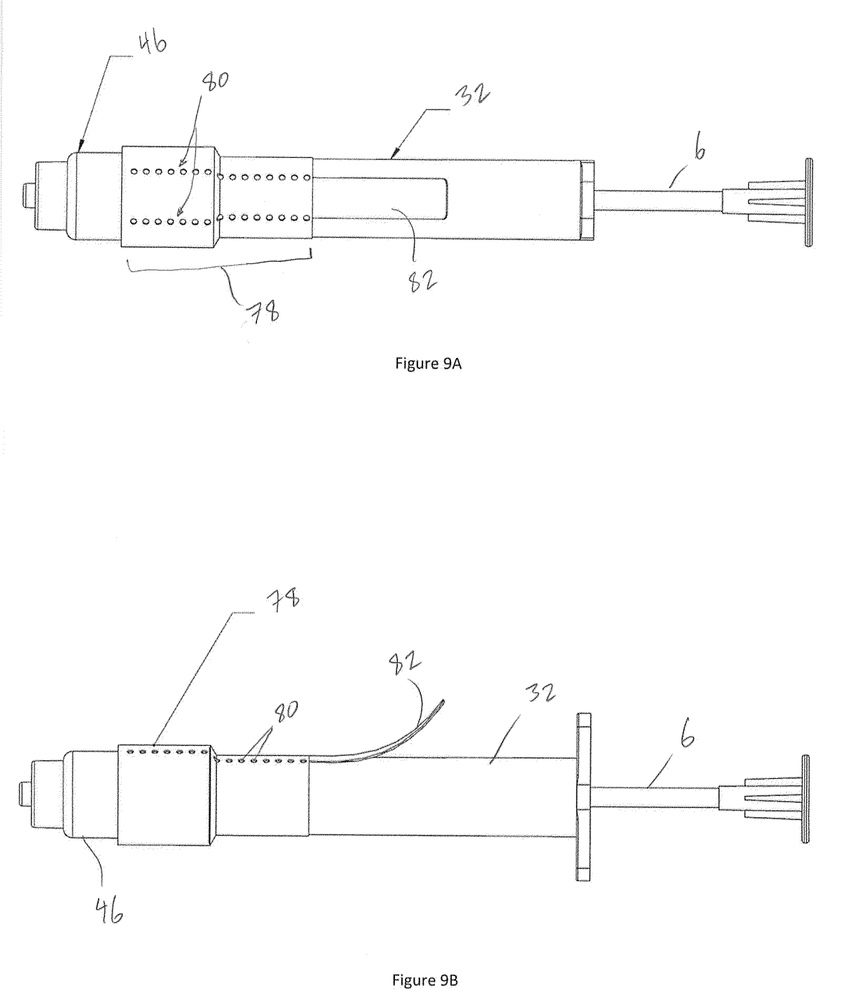

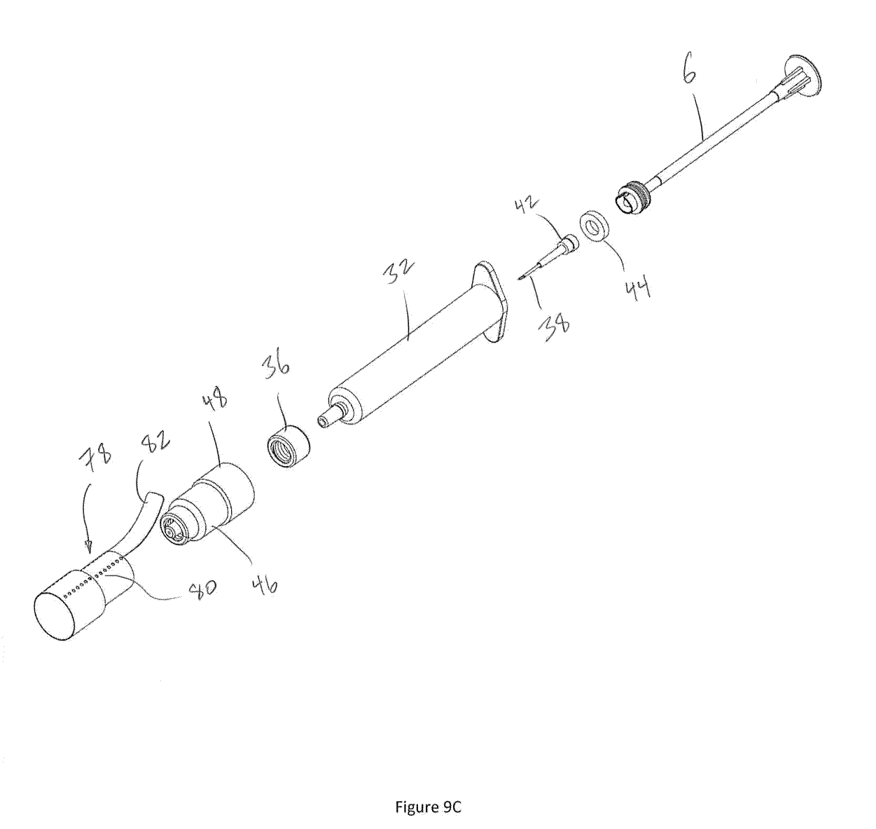

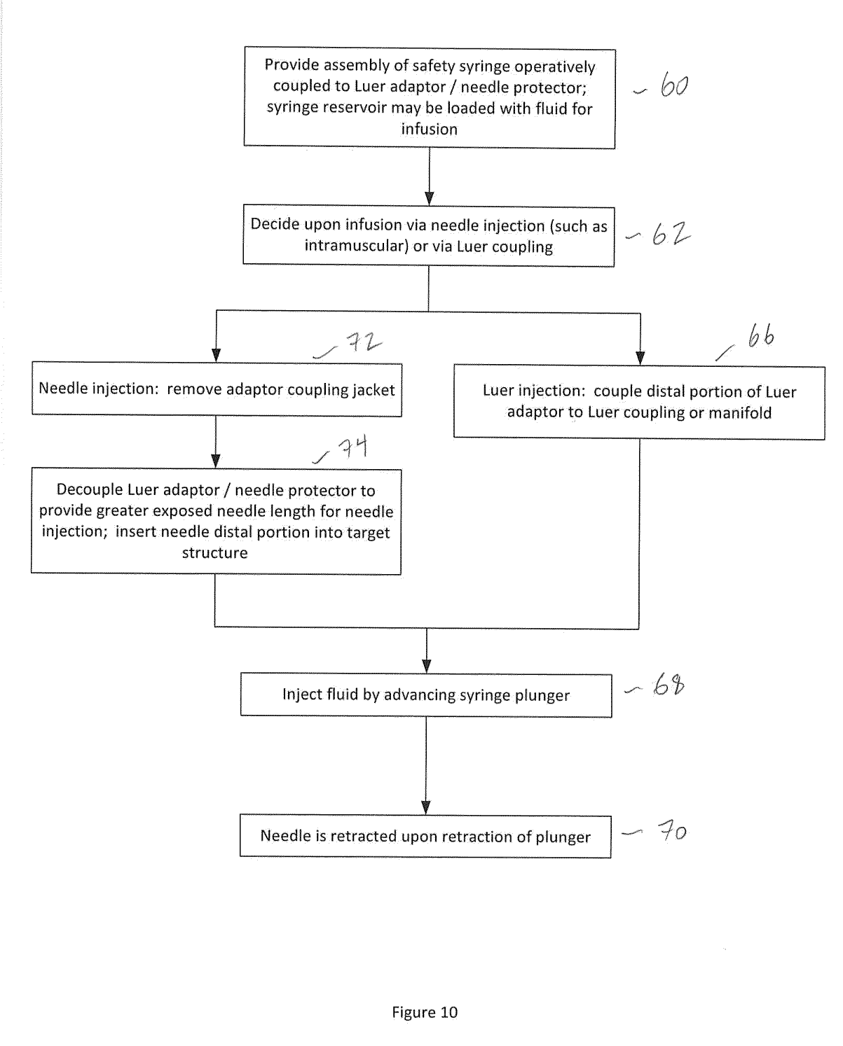

Referring to FIG. 10, a process embodiment similar to that of FIG. 8 is depicted, with the exception that in a needle injection configuration (72, 74), prior to decoupling the Luer adaptor from the remainder of the syringe assembly, the adaptor coupling jacket is removed by the operator.

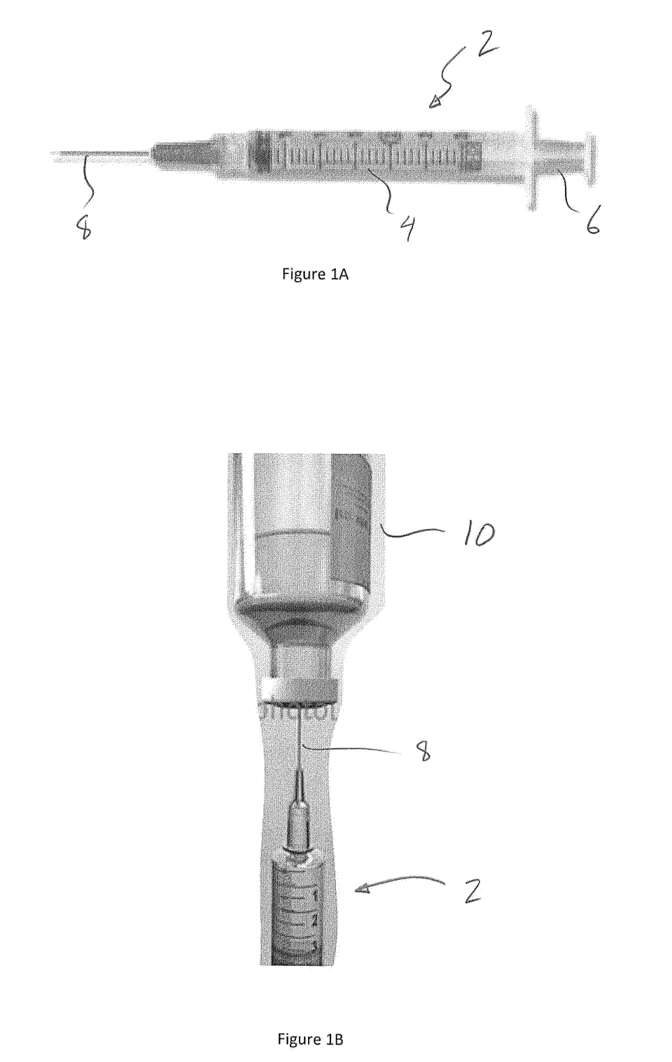

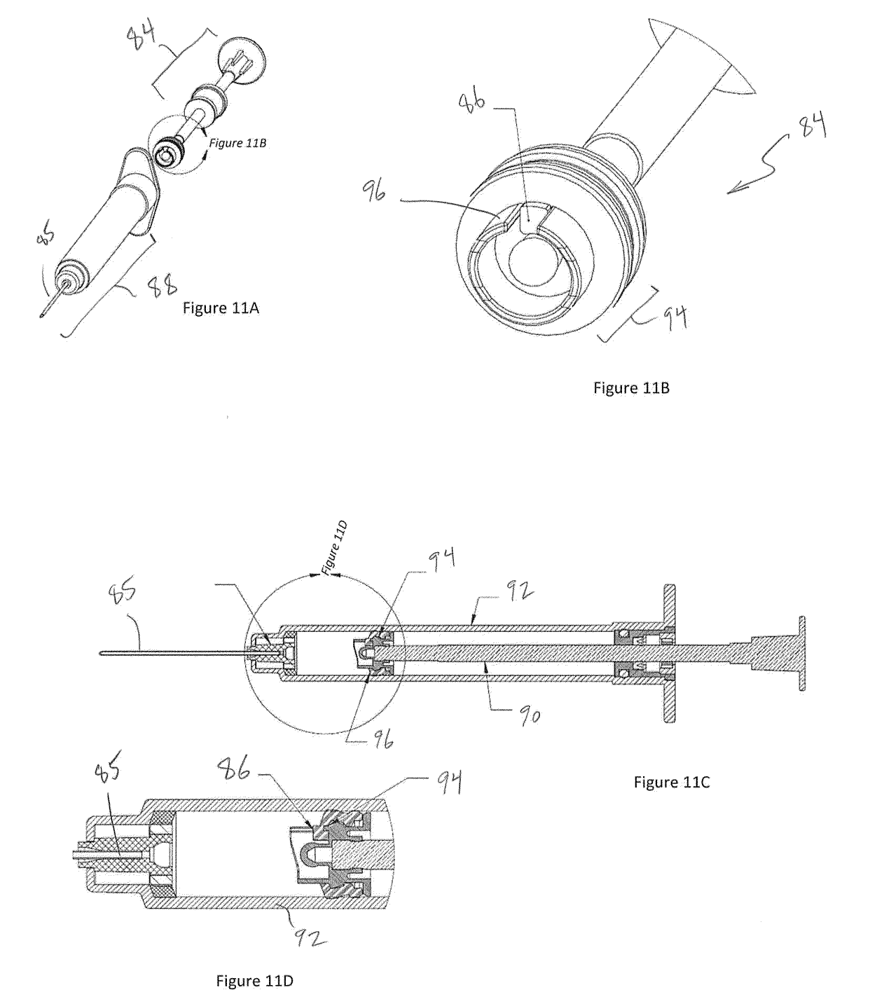

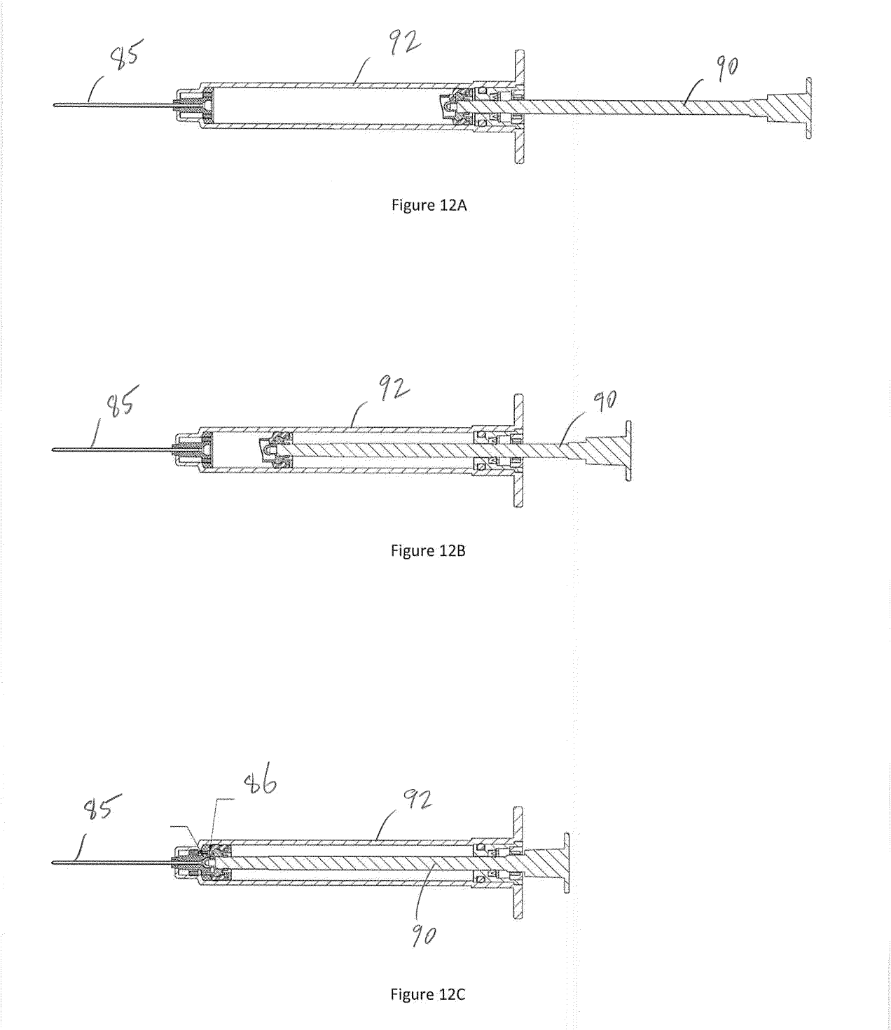

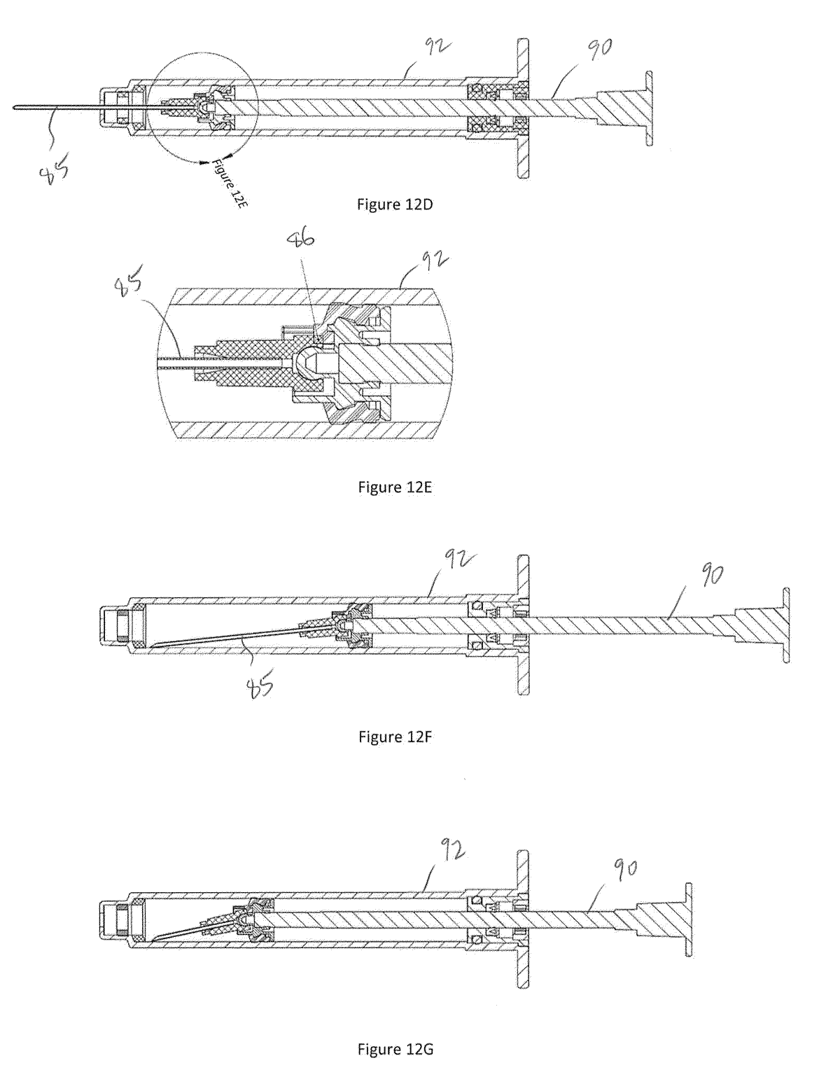

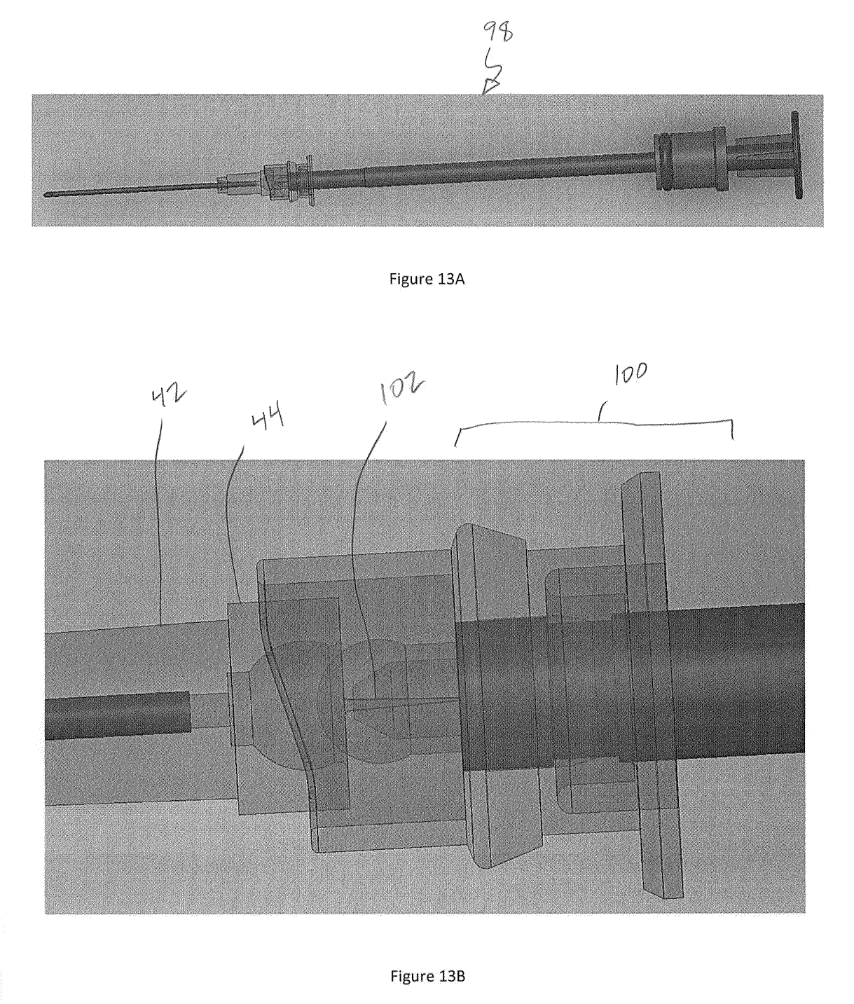

Referring to FIGS. 11A-14, various configurations are illustrated that are applicable not only to configurations such as those described in reference to FIGS. 1A-10, but also to various other styles of safety syringes involving needle member retraction after plunger insertion, with or without Luer adaptor configurations. Referring to FIGS. 11A-12G, in one embodiment, after full insertion of a plunger assembly (84) relative to a syringe body/needle assembly (88), an intentional misalignment or shim member (86) may be provided at the mechanical interface between the needle and the plunger that causes the needle member (85) to reorient itself away from the axis of previous insertion when the plunger is fully withdrawn, thereby allowing the needle member (85) to have axial freedom of motion. Referring to FIG. 11B, the shim member (86) may comprise a portion of elastomeric material formed from an interruption in the geometry of a forward-oriented portion (96) of the plunger seal (94)—as shown in FIG. 11B wherein the shim member (86) comprises a flapped over portion that will be compressed and biased to re-orient the needle member when the plunger shaft (90) is inserted to a full insertion position relative to the syringe body (92). FIGS. 12A-12G show a sequential progression. Referring to FIG. 12A, a full syringe is ready for injection. FIG. 12B shows partial insertion of the plunger shaft (90), while FIG. 12C shows full insertion of the plunger shaft (90) and thereby coupling of the plunger assembly to the needle assembly (for example, as described above)—with the shim member (86) in compression and biased to misalign the needle relative to the axis of insertion should the needle member be free to deflect/reorient. FIG. 12D shows partial retraction with the needle still following along the axis of needle insertion by virtue of the mechanical constraints of the distal syringe body componentry; FIG. 12E shows that the shim member remains in compression, biased to reorient the needle should the needle have less constraint. Referring to FIG. 12F, with the end of the needle withdrawn fully past the end of the constraining syringe body structure, the shim member (86) causes the needle to reorient and stay reoriented, so that even with insertion forces applied against the plunger shaft, the needle generally will be biased to stay within the safe confines of the syringe body, as shown. FIG. 12G illustrates a similar configuration for a shorter needle member. The configuration of FIG. 12F is approximately illustrative of a 30 mm long needle reoriented by 6 degrees; the configuration of FIG. 12G is approximately illustrative of a 10 mm long needle reoriented by 12 degrees. In one embodiment, it is desirable for a reorienting element that has a relatively low spring constant such that it will work to reorient both long and short needles into configurations such as those depicted in FIGS. 12F and 12G.

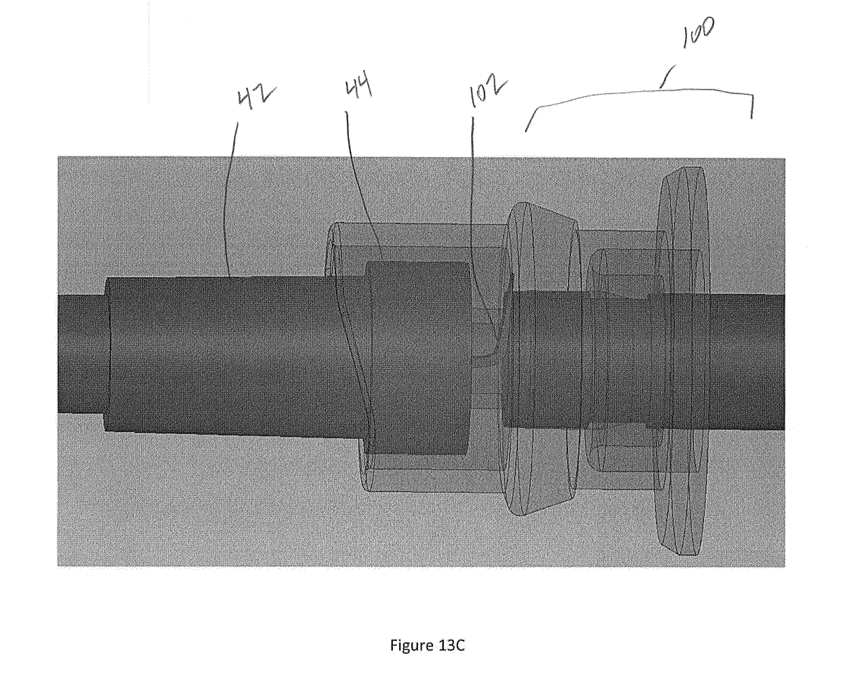

Referring to FIGS. 13A-13C, a cantilever bending reorientation member (102) coupled to the needle member or plunger member (in the depicted embodiment, coupled to the needle member) may serve a similar purpose as the shim member (element 86) of FIGS. 11A-12G. A partial safety syringe assembly (98) is shown in FIG. 13A, and close-up in FIGS. 13B and 13C to illustrate that upon coupling/interfacing of a plunger assembly (100) and a needle assembly (needle holder 42 and interfacing grommet 44 are shown), such as by full insertion of the plunger assembly relative to a syringe body (not shown), the cantilever bending reorientation member (102) may be placed into bending, as shown in FIG. 13C, such that upon full withdrawal of the plunger assembly and now-intercoupled needle assembly past the orientation constraints which may be associated with the syringe body or housing, stored energy in the bent cantilever bending reorientation member (102) will cause the needle member to reorient and generally become resistant to further insertion back out of the syringe body, in a similar manner as illustrated in FIGS. 12F and 12G for the shim member configuration.

The spring constant of the reorienting member may be modulated by changing not only the material comprising such element, but also the geometry (i.e., length, cross sectional shape, cross sectional area versus length, etc). For example, in one embodiment with an elastomeric compression reorienting member (86), a pyramidal cross sectional geometry may be utilized to produce a nonlinear profile of bending/reorienting loading which does not significantly interfere with plunger insertion or needle capture.

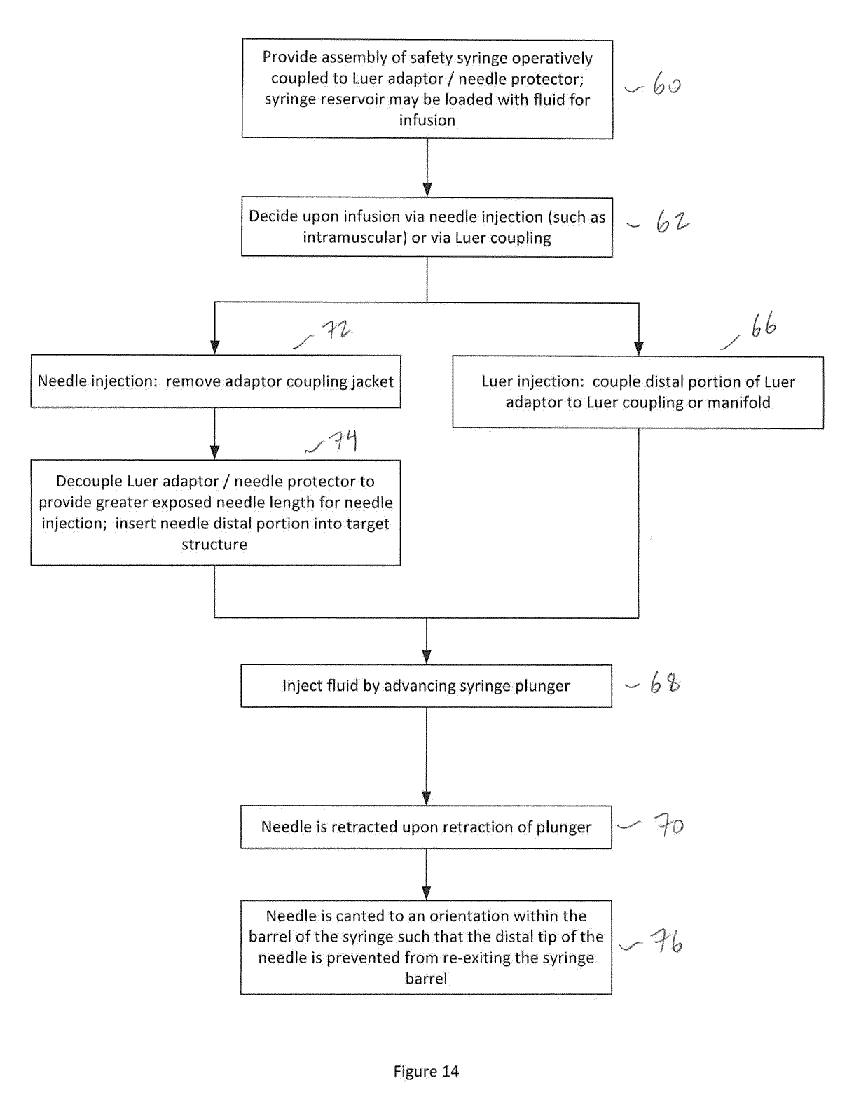

Referring to FIG. 14, a process embodiment similar to that shown in FIG. 10 is depicted, with the addition that upon retraction of the needle past the distal alignment constraints, such as those which may be provided by the syringe body, the needle is “canted” or reoriented so that it will be prevented from re-exiting the syringe body.

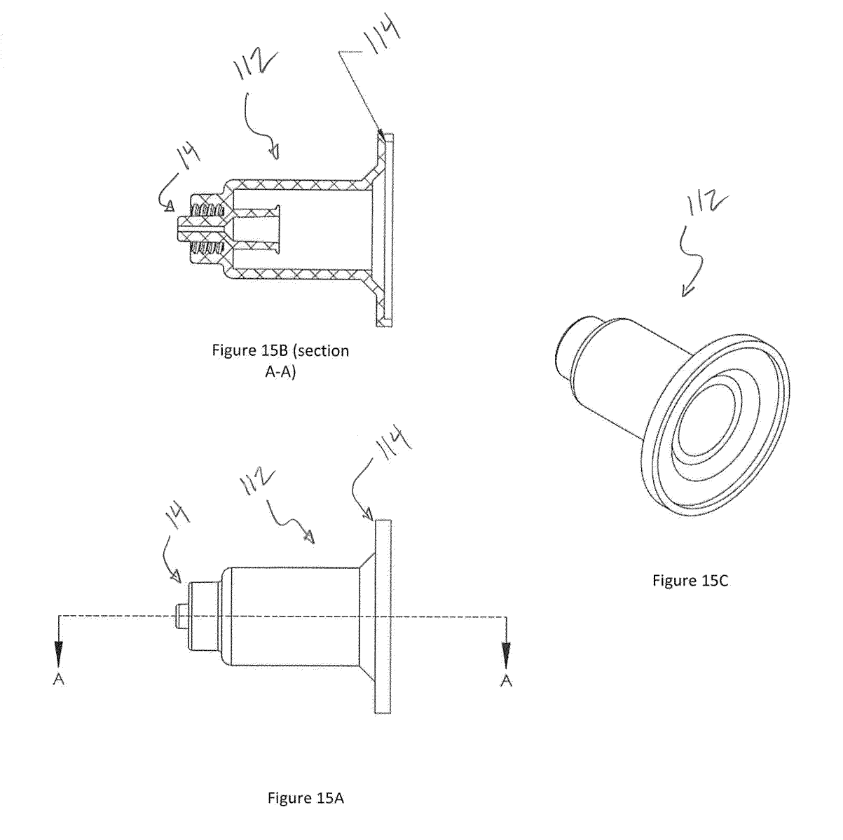

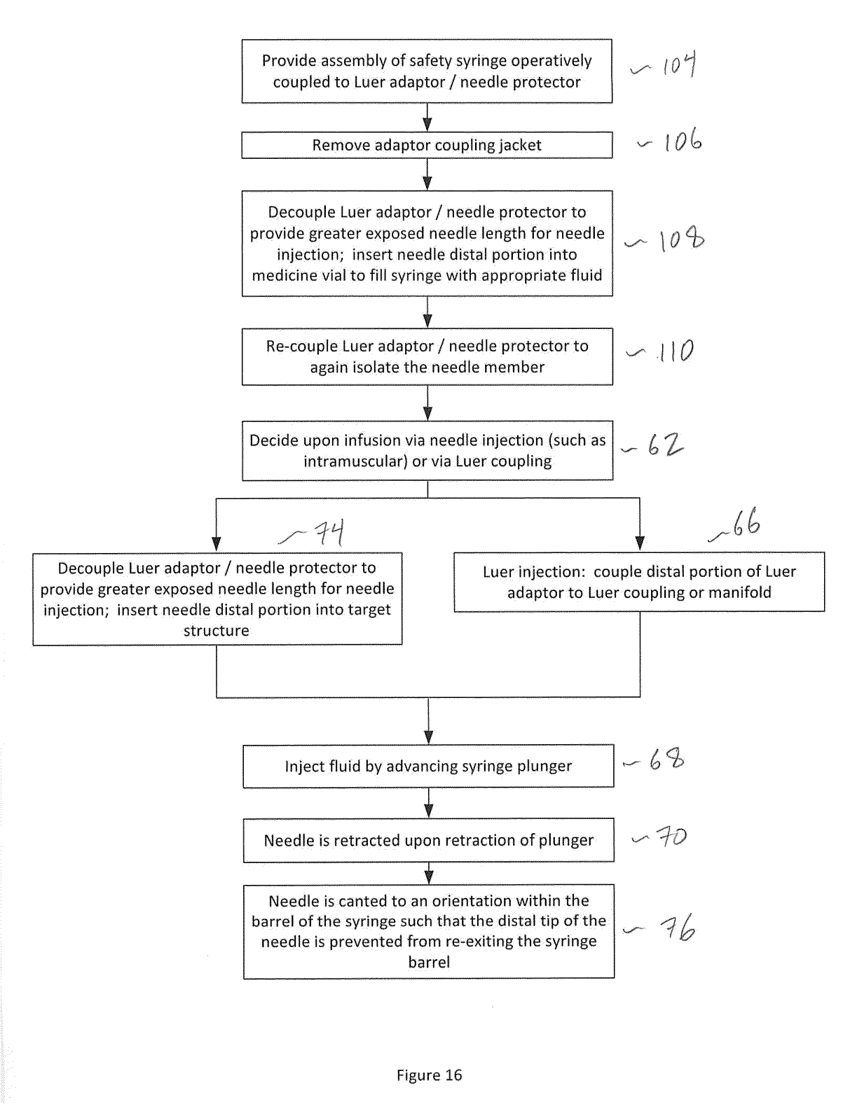

Referring to FIGS. 15A-15C, another embodiment of a Luer adaptor member (112) suitable for use with configurations such as those shown in FIGS. 6A and 7A (i.e., in replacement of adaptors 46 and 50, respectively), for example, is illustrated in three views. This embodiment (112) comprises a flange (114) configured to be protective of the fingers of an operator when the adaptor member (112) is being re-coupled to the syringe assembly, such as in a configuration as described below in reference to FIG. 16.

Referring to FIG. 16, a process embodiment somewhat similar to that shown in FIG. 14 is depicted, with a filling process before usage. As shown in FIG. 16, an assembly of a safety syringe coupled to a Luer adaptor is provided (104). The adaptor coupling jacket may be removed (106), along with the Luer adaptor (108) so that the syringe may be filled using the conventional technique of inserting the needle into a medicine vial and pulling back the plunger. In one embodiment the plunger should not be fully inserted before withdrawal so that the needle does not become coupled to the plunger and withdrawn into the syringe body prematurely before the remaining steps outlined herein. With the syringe filled with the appropriate fluid (108), the Luer adaptor may be re-coupled to the syringe assembly to again isolate the needle member (110); in one embodiment a flanged Luer adaptor, such as that illustrated in FIGS. 15A-15C, may be utilized to assist with protecting the fingers of the operator during such re-coupling step. At such point, with a filled syringe and Luer adaptor in place, the steps are similar to those described in reference to FIG. 14, with the exception that the adaptor coupling jacket has already been removed (element 106—described above).

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

1. A method of conducting an injection, comprising:
a. providing an assembly of a syringe plunger movably coupled to a syringe body which may contain fluid for infusion, the syringe body having a distal end that is removably coupled to a Luer adaptor, the Luer adaptor defining a needle lumen through which a needle may be coupled, the Luer adaptor comprising a distal surface configured to be removably coupled to a Luer fitting of another device;
b. deciding upon infusion via needle injection or Luer coupling; and
c. selectably configuring the assembly for needle injection or Luer coupling by removing the Luer adaptor to further expose the needle for needle injection, or by leaving the Luer adaptor in place and coupling it to the Luer fitting of the other device.

2. The method of claim 1, further comprising inserting the plunger relative to the syringe body to inject fluid into an infusion target.

3. The method of claim 2, wherein Luer coupling is selected, the method further comprising rotatably coupling the distal surface of the Luer adaptor to the Luer fitting of the other device.

4. The method of claim 2, wherein needle injection is selected, the method further comprising inserting a portion of the needle directly into a patient.

5. The method of claim 1, further comprising coupling a proximal end of the needle to the plunger distal end upon full insert of the plunger relative to the syringe body.

6. The method of claim 5, further comprising reorienting the needle upon full retraction relative to the syringe body to avoid re-insertion of the needle relative to the syringe body.

7. The method of claim 6, wherein full retraction is reached by pulling the plunger relative to the syringe body.

8. The method of claim 7, wherein the state of full retraction is assisted via a vacuum load developed within the syringe body upon insertion of the plunger relative to the body.

9. The method of claim 1, wherein removing the luer adaptor comprises removing an outer coupling jacket configured to retain coupling between the Luer adaptor and the syringe body.

10. The method of claim 9, wherein removing the outer coupling jacket comprises manually manipulating a portion of the outer coupling jacket that is pre-biased to tear upon loading for easy removal.

11. A safety syringe system, comprising:
a. a plunger movably coupled to a syringe body, the syringe body having proximal and distal ends; and
b. a Luer adaptor removably coupled to the distal end of the syringe body and defining a needle lumen through which a needle may be coupled, the Luer adaptor comprising a distal surface configured to be removably coupled to a Luer fitting of another device;
wherein the needle has proximal and distal ends, the distal end configured for direct injection into an injection target structure, the proximal end configured to be coupled to the plunger such that upon withdrawal after insertion, the needle is pulled into and contained by the syringe body.

12. The system of claim 11, wherein the syringe body comprises a glass construct.

13. The system of claim 11, wherein the syringe body comprises a polymeric construct.

14. The system of claim 11, wherein the distal surface comprises a Luer mating geometry.

15. The system of claim 14, wherein the other device may be removably coupled to the distal surface by inducing relative rotational motion between the other device and the distal surface.

16. The system of claim 11, wherein the distal end of the needle comprises a hypodermic needle tip.

17. The system of claim 11, wherein the proximal end of the needle is configured to be compressibly coupled to the plunger.

18. The system of claim 11, further comprising an outer coupling jacket configured to retain the coupling between the Luer adaptor and the syringe body when in place.

19. The system of claim 18, wherein the outer coupling jacket is removably coupled across portions of both the Luer adaptor and the syringe body.

20. The system of claim 19, wherein the outer coupling jacket comprises a heat shrink tubing member.
